(19) United States
(12) Patent Application Publication
(10) Pub. No.: US 2023/0039246 A1
(43) Pub. Date: Feb. 9, 2023

(54) NON-BRAIDED BIODEGRABLE FLOW DIVERTING DEVICE FOR ENDOVASCULAR TREATMENT OF ANEURYSM AND ASSOCIATED FABRICATION METHOD

(71) Applicant: University of Central Oklahoma, Edmond, OK (US)

(72) Inventors: Mohammad Robiul HOSSAN, Edmond, OK (US); Seth HARRIET, Edmond, OK (US); Andrew M. BAUER, Jones, OK (US)

(21) Appl. No.: 17/871,001

(22) Filed: Jul. 22, 2022

Related U.S. Application Data
(60) Provisional application No. 63/230,409, filed on Aug. 6, 2021.

(51) Int. Cl. A61B 17/12; B33Y 80/00; B33Y 30/00; B29C 64/118; B29C 64/393; B33Y 50/02

(52) U.S. Cl. CPC A61B 17/12113; A61B 17/12131; B33Y 80/00; B33Y 30/00; B29C 64/118; B29C 64/393; B33Y 50/02; A61B 2017/00004

(57) ABSTRACT

A biodegradable flow diverting device (BFDD) that will regulate blood flow into an aneurysmal sac, act as a scaffold for endothelization at the neck of an aneurysm, and degrade after successful dissolution of aneurysm and remodeling of blood vessel. This BFDD and associated fabrication method have the following features: (1) This is a non-braided FDD. The pore shapes, sizes, architectures (especially at the inlet and outlet of the pores), pore densities and porosities can be controlled for the optimum performance depending on the blood vessel and aneurysmal morphologies from patient MRI images, (2) BFDD is developed on a rotary arm with programmable variable speed and diameter in conjunction with a micromotion stage (3) Fabrication system can take any material including blended/composite biomaterials by adjusting temperature of the electro-melt extruder/needle and (4) Fabrication system is compatible with CAM (computer aided manufacturing) software and able to operate based on the adapted G-code.

### STATEMENT AS TO RIGHTS TO INVENTIONS MADE UNDER FEDERALLY SPONSORED RESEARCH OR DEVELOPMENT

This invention was made with government support under Grant Number 8P2OGM103447 awarded by the National Institutes of Health. The government has certain rights in the invention.

### FIELD OF THE INVENTION

The present invention generally relates to medical devices intended for endovascular treatment of aneurysms and methods for fabrication thereof. More specifically, the present invention relates to a non-braided biodegrable flow divertering device for endovascular treatment of aneurysm and associated modular fabrication method.

### BACKGROUND OF THE INVENTION

Aneurysm is a localized dilation of the vascular wall that can make it to swell out beyond control and rupture if not treated. In the United States, 15% of aneurysmal subarachnoid hemorrhages (SAH) patients die before they reach the hospital, 66% of survived patients suffer from a permanent neurological deficit, and 57% have disabilities. How-diverter devices (FDDs) are new-generation stents placed in the parent artery at the level of the aneurysm neck to disrupt the intra-aneurysmal flow, providing significant hemodynamic effects with potential changes in biological and biochemical responses. They progressively create intra-aneurysmal thrombosis, offering good support for the development of a neointima. Upon approval from FDA in 2011 (for limited and specific use) and in 2018 (3^{rd} generation), fine mesh/braided metallic flow diverters have shown promising clinical outcome in aneurysm treatment. However challenges in overall aneurysm treatment such as late thrombosis, rupture, non-IA related intracerebral hemorrhages (ICH), incomplete occlusion, higher total complication and mortality rate are still unresolved.

Five types of FDDs have been approved for the treatment of intracranial aneurysms: the Pipeline Embolization Device (PED) (Covidien, Mansfield, Mass., USA), the Silk (Balt Extrusion, Montmorency, France), the Flow Re-direction Endoluminal Device (FRED) (Microvention, Tustin, Calif., USA), the p64 Flow-Modulation Device (Phoenix, Ariz., USA) and the Surpass Flow-Diverter (Surpass; Stryker Neurovascular, Fremont, Calif., USA). All of the forgoing FDDs are mesh/braided designs; none are bioresorbable. Current mesh/braided FDDs are very limited in controllable design parameters such as: a) braided FDD design is limited to diamond shaped pores, b) the braided FDD design has no room for surface engineering, architectural and topographical change that can regulate blood flow in and out from the aneurysm differently for engineered thrombus formation inside aneurysm, reduce the chance of distant embolism formation and travel of embolisms from the aneurysmal sac, and c) current FDD are based on and limited to metallic alloys and are not bioresorbable. In addition, there is no method to produce continuous microfiber thread with appropriate mechanical properties and surface quality from bioresorbable material that can be used in current FDD braiding machines to produce bioresorbable braided FDDs. Coronary scaffolds which fully degrade (for example Absorb Bioresorbable Vascular Scaffold System (BVS), Abbott Vascular, Santa Clara, USA) have been introduced in the past decade. A BVS is a non-metallic mesh tube that is used to treat a narrowed artery, is similar to a stent, but slowly dissolves once the blocked artery can function naturally again and stays open on its own. The bioresorbable and drug eluting stents show clinically less inflammatory and improved healing, but are not functional as a FDD. The shortcomings of mesh/braided FDDs can be resolved with the present invention, providing a needed bioresorbable and non-braided flow diverter with more controllable FDD design parameters.

### SUMMARY OF THE INVENTION

The present invention provides bioresorbable flow diveter devices (FDDs) that are different in terms of their purposes, functionality, and structure. While presently available FDDs are mesh/braided devices, the FDD of the present invention is non-braided. Further, most bioresorabable stents are porus shapes made by laser cutting, and all of these devices differ in their physical structure. The fabrication methods of the prior art are different from the fabrication method of the present invention and produce a different result. The present invention provides a fabrication method that exploits fused deposition model (FDM) based 3D printing technology. The present invention provides a fabrication method wherein the FDD is made on a precisely controlled rotary arm. Further, blended materials may be incorporated in powder form and solvent-based extrusion added which are not currently available in FDM 3D printing technologies. The both rotational speed of the rotary arm and the micromotion stage movement have significant impact on the bioresorbable FDD properties and designs. In the present invention, the rotation speed of the rotary arm determines the bioresorbable FDD strut size, while the micromotion stage movement controls the porosities and pore shapes of FDD. Also, current FDM and resin 3D printers (DLP or SLA) use CAD slicing softwares, where the method of the present invention is based on progamming codes, and the codes are generated by a novel mathematical relationship provided by the present invention. Based on the desired porosities of the bioresorbable FDD, the mathematical relation determines the rotational speed of the rotary arm, the translational motion of the micromotion stage and number of passes etc. Also, based on this mathematical determination, the fabrcation system generates necessary G-codes and automatically begin fabrication of the bioresorbable FDD.

G-code (also known as RS-274) is the name of the most prevalent programming language for computer numerical control (CNC) in computer-aided design and manufacturing (CAD/CAM) the G-code provides metric-based numeric control of CAM-controlled equipment such as CNC milling machines. The fine-grained control enabled by G-code and other CNC languages afford the precision for additive and reduction-based fabricating using many materials.

In some embodiments of the present invention, the fabrication system can be opted to produce continous bioresorbable smooth microfiber with high surface quality. ASME Standard B46.1, incorporated herein by reference, describes surface roughness as the average of peaks and valleys across a surface. Surface roughness can be measured by surface profiling instruments The measure is expressed as arithmetic average (Ra) in microns. The present invention can produce microfiber with surface characteristics exhibiting an Ra below 0.12 micrometers (microns) where samples have been measured to exhibit Ra at 0.1186 and 0.0752 micrometers. Depending on the desired microfiber diameter, a developed mathematical relation determines the rotational speed of the rotary arm and necessary tension force, generate necessary G-codes and operate the fabrication systems. Adjustment of these parameters significantly influence surface quality of the microfiber.

The competing fabrication technology of making microfiber is electrospinning, which requires strong electric fields. In contrast, the modular fabrication method of making microfiber provided by the present invention, requires no electric field and spinning is not used. In the present invention, the electro-melt extruder nozzle, precision tension pulley and cooling systems are used to produce micrometer fiber. Tension force and cooling are combinedly utilized inputs to control the microfiber diameter. Also electrospin fibers are not uniform in their size and surface quality are rough. In contrast, the present invention produces extremely uniform microfiber that exhibit a very smooth surface, where Ra is less than 0.12 micrometer. Microfiber from the electrospinning are not suitable for using as surgical suture and making braided bioresorbable flow diverters, while the microfiber of the present invention are suitable for making braided bioresorbable flow diverters and/or as surgical suture.

In some embodiments of the present invention, necessary G-codes are generated from the developed mathematical expression and operation of the fabrication system can be automated.

The present invention provides flow diverter devices and associated modular fabrication methods that are novel over the prior art because 1) the FDD of the present invention is biodegradable, not permanent in the body; 2) the FDD of the present invention is not braided, so pore shapes and sizes, mechanical properties of the FDD, surface architecture, roughness and texture can be optimized for better clinical outcomes; 3) the fabrication method is robust in terms of automation and flexibility, exploits and combines the features from fused deposition model tecniques and spinning technolgy.

In one aspect, the present invention provides a modular fabrication method to produce (i) continuous fiber/thread with micrometer diameter from bioresorbable (and non-bioresorbable) material usable in surgical sutures or flow diverters.

In another aspect, the bioresorbable FDD of the present invention may comprise Poly(ϵ-caprolactone) (PCL), however it may be extended to comprise other biodegradable materials, and composite/blended biodegradable materials. Hence it will not cause persistent low-grade inflammation for indefinite time, and it will reduce chances of micromotion and injury since it will be absorbed by the body.

In another aspect, the fabrication method may produce the FDD on a rotary arm that ensures mechanical integrity of the FDD during formation.

In another aspect, the fabrication method may be converted to 3D print on a flat surface;

In another aspect, the fabrication method is compatible to CAD/CAM software that produce necessary G-codes for fabrication.

In one aspect, the fabrication method includes a modular adjustment (removeable precision tension pulley with cooling system) capable of producing continuous thread/microfiber from bioresorbable material.

In another aspect, continuous thread/microfiber is collected on a spool.

In another aspect, the fabrication method of the present invention can control the mechanical strength, performance and degradation rates of bioresorbable microfiber suture by altering diameter sizes, incorporating anti-bacterial, anti-inflammatory and other biomaterial coatings.

In another aspect, the fabrication method of the present invention can continually produce microfiber exhibiting an average surface roughness (Ra) less than 0.15 micrometer.

The foregoing paragraphs provide a general introduction, and are not intended to limit the scope of the claims presented herein. The described embodiments, together with further advantages, are best understood by reference to the following detailed description taken in conjunction with the drawings presented. Below, the embodiments as described in detail will make easily understood the objectives, technical contents, characteristics and accomplishments of the present invention.

### BRIEF DESCRIPTION OF THE DRAWINGS

FIG. 1 is a non-limiting diagram schematically showing the fabrication unit capable of producing the bioresorbable flow diverter device (FDD) of the present invention.

FIG. 2 is a non-limiting diagram schematically showing the fabrication unit of the present invention configured to produce continuous micrometer fiber from bioresorbable material and collect the fiber on a spool.

FIG. 3 is a non-limiting diagram schematically showing the hybrid modular fabrication system of the present invention for making bioresorbable flow diverting device (BFDD) and continuous micrometer fiber from bioresorbable material and collected on a spool on demand.

FIG. 4 is a non-limiting diagram schematically showing an alternate shape of the bioresorbable flow diverting device (BFDD) fabrication unit configured for upright flatbed systems.

FIG. 5a is a non-limiting diagram schematically showing the elements comprising the flow diverter device (FDD) as a cylindrical tube according to one embodiment of the present invention.

FIG. 5b is a non-limiting diagram schematically showing the elements comprising the flow diverter device (FDD) as a globe shaped object according to another embodiment of the present invention.

FIG. 6 is a non-limiting diagram showing the non-braided surface structure of the FDD provided by the present invention.

FIG. 7 is a non-limiting diagram showing an embodiment of the present invention placed in a parent artery at the level of the aneurysm neck to disrupt the intra-aneurysmal.

FIG. 8, is a non-limiting diagram showing a process for fabricating the FDD of the present invention using the fabrication unit (FIG. 1-100, FIG. 2-200, FIG. 3-300) of the present invention.

### DETAILED DESCRIPTION OF THE INVENTION

In Brief: The present invention provides a flexible, highly porous (50% to 80%) flow diverter device (FDD) with various pore shapes and sizes as a cylindrical tube and globe shaped object based on PCL that can regulate blood flow inside an aneurysmal sac. The both cylindrical and globe shaped BFDD can be delivered to the appropriate vascular region through common endovascular surgical catheter.

The present invention provides a method of fabricating the non-braided FDDs. The fabrication unit includes a rotary arm (variable diameter) controlled by motors, integrated and electronic circuitries and programming codes, X-Y-Z micromotion stages, feeding gear, temperature controlled electro-melt extruder nozzle/needle (variable diameter) and cooling fan. The input of the system includes diameter of the tube, design of the pores (shape, size and pore density, microfiber diameter (for continuous fiber production)) through programming codes, rotary arm rotation speed, feeding rate and cooling rate.

The flow diverters are designed by sketching and converted into programming codes for X-Y and rotary movement. Appropriate rotary arm is selected and put into the motor chuck. The extruder nozzle diameter, material (here PCL), melting temperature, feed rate and cooling fan speeds can be selected as input. After having all necessary inputs, the fabrication unit starts fabricating a flow diverter on the rotary arm and it stops when fabrication is complete. The fabricated FDD is taken out from the rotary arm and coated with BaSO₄ (for visibility in angiogram during delivery to the body) or Bismuth Nitrite or other radiopaque coating, and then it is ready to use for endovascular treatment.

For production of continuous on-demand bioresorbable microfiber and collected on a spool, the micro-motion stage is replaced with the precision tension force-based pulley and cooling system. With precision tuning of tension force and cooling fan, the microfiber with the desired diameter is produced from the electro-melt extruder nozzle and collected in the spool. The produced continuous microfiber (in this case, PCL) is flexible and can be used in making braided cylindrical or globe shaped flow diverters.

The present invention provides composite biodegradable material-based FDDs. PCL/PLA or other biomaterial may be blended with other biomaterials, angiogenic factors/healing accelerator/inflammation reducing ingredients for faster post-surgery healing, improving endothelization, localizing thrombus formation inside the aneurysm etc.

The fabrication unit is adapted to accept a liquid or powder form of PCL and other biomaterial, and provide solvent-based extrusion. The fabrication unit is also adapted to use PCL, PLA and Bioflex antibacterial filament which is available for commercial 3D printers.

The fabrication system of the present invention is modular, which means the system is easily convertible to produce continuous, uniform and smoother bioresorbable microfiber and is collected on a spool. The produced continuous microfiber filament can be used according to the method of the present invention to fabricate bioresorbable braided flow diverters of different types including cylindrical for aneurysms on a blood vessel and globe shaped flow diverters for the aneurysm on a blood vessel junction. The continuous microfiber from the fabrication system of the present invention can be used as surgical suture as well. The microfiber of the present invention can receive BaSO₄ and Bismuth Trioxide coating for the x-ray visibility under angiographic intervention. The FDD and microfiber suture of the present invention may also be coated with anti-inflammatory ingredients and antibacterial agents.

In terms of functionality, the modular fabrication system and bioresorbable flow diverters provided by the present invention are different in at least the following aspects: 1) metallic FDDs are permanent in the body, in contrast, the PCL FDD of the present invention will be absorbed by the body after complete dissolution of aneurysms, reducing the possibility of future complication; 2) a Metallic FDD does not allow re-intervention if needed and interfare with the MRI/CT scan or electro-magnetic field based diagnostics, while the PCL FDD of the present invention will allow re-intervention and will not have intefereance with electro-magnetic field based diagnostics; 3) the PCL FDD design of the present invention provides more controllable parameters to regulate blood flow into the aneurysmal sac, while currently available FDD designs are limited due to braided design; 4) the fabrication method of the present invention provides PCL FDDs with various sizes and shapes such as cylinderical, globe shaped and irregular shaped (i.e. variable nominal diameters and shape) to fit into the complex blood vessel networks (like aneurysm in complex blood vessel junctions), while current FDD designs are limited to cylinderical shape; 5) the modular fabrication system of the present invention can produce continuous, uniform, smooth and spoolable PCL microfiber on-demand with precise diameters/sizes (through precision tension force pulley and cooling system), while current electro or other spinning method cannot produce continuous, uniform and smooth PCL microfiber with on-demand precise diameter, and 6) unlike currently available systems, the fabrication system of the present invention exploits and combines spinning technolgy and fused desposition modeling into one hybrid fabrication systems for biomedical and engineeirng applications.

Current FDM based 3D printing technology uses 3D CAD slicing software to generate a tool path for the extrusion nozzle and model developed on a heated bed. In the fabrication method of the present invention, the extrusion nozzle remains fixed, the motion stage moves and an FDD is made on a rotary arm. The rotation speed of the rotary arm is one of the most significant process parameters for the BFD of the present invention. The rotary arm is used to control FDD design by modulating rotary speed.

In structural aspect, 1) the current or available FDDs are based on braided microwires, while the FDD of the present invention is non-braided; 2) The PCL FDD of the present invention is a biodegradable material, on the other hand the current FDD materials are not biodegradable and are based on metallic wires; 3) pore shape and design are not limited to diamond shapes like in the current braided FDDs; 4) current braided FDDs use platinum wire for the radiopaquacity, while the BFD of the present invention is coated with BaSO4 or Bismuth trioxide for radiopaquacity; and 5) current PCL fiber producing machines use electric field and spinning technolgy, while the method of the present invention provides a precision tension pulley and cooling system, and does not use an electric field.

In the fabrication unit of the present invention, 1) the electro-melt extruder remains fixed, i.e. does not have x and y movement, while in conventional FDM 3D printer methods, the extruder moves in all 3 directions, i.e. x, y, z; 2) the FDD of the present invention can be made on the rotary arm, while in the FDM system the FDD is build on a flat bed; 3) the tension pulley and cooling fan of the present invention are vital components for producing continuous, smooth and unifrom spoolable microfiber, while spinning techology uses electric field and spinning drum produce fiber which lack in controllable parameters.

### In Detail:

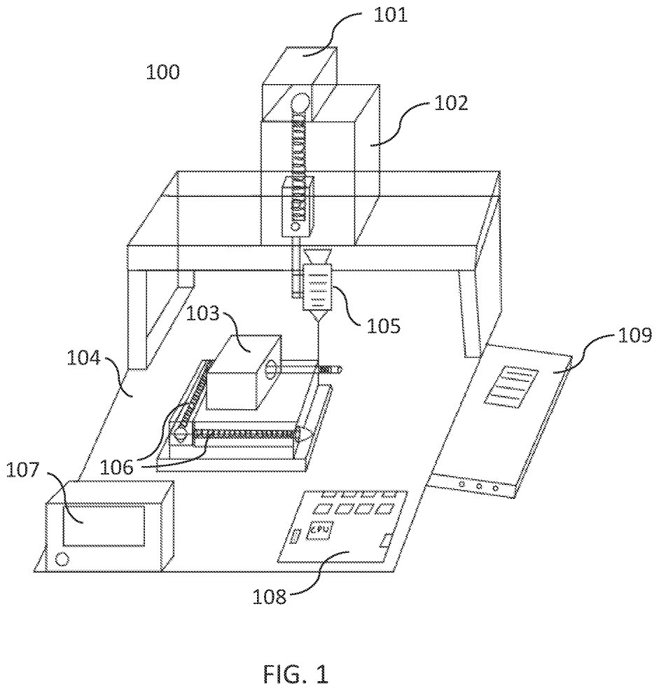

Referring to FIG. 1 a non-limiting diagram illustrates schematically the fabrication unit 100 of the present invention making a bioresorbable flow diverter device (FDD). Power screw drive system 101 as shown includes Z-axis actuation 102 which provides for vertical positioning, the rotary arm 103 may include a variable chuck that can rotate either direction and take different shape/size rotary arms. The rotary arm 103 is also used to control quality of surface characteristics of bioresorbable FDD. The fabrication system is built on aluminum frame 104. The replaceable electro-melt extruder 105 may include a connected feed material container. The electro-melt extruder 105 can take filament as well as powder/bead form of material to feed through the electro-melt extruder 105. The micromotion stage 106 has two degrees of freedom and it allows the stage to move in x direction and y direction. Control box 107 and CPU 108 work as user interface where input parameters are fed into the fabrication unit 100. The fabrication unit 100 is powered by the 12-volt power supply 109.

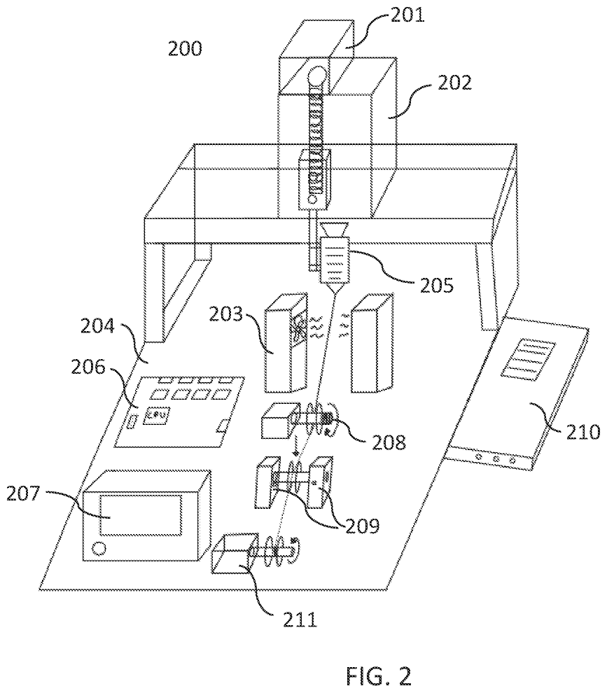

Referring to FIG. 2, a non-limiting diagram illustrates schematically the fabrication unit 200 (FIG. 100) for producing continuous micrometer fiber from bioresorbable material and collected on a spool. The power screw drive system 201 as shown provides Z-axis actuation 202 for vertical positioning. The cooling system 203 incorporates an integrated fan and temperature sensors. The fabrication unit 200 (FIG. 100) includes an aluminum frame 204. The replaceable electro-melt extruder 205 with connected feed material container can take filament as well as powder/bead form of material to feed through the electro-melt extruder 205. The control box 206 and CPU 207 work as user interface where input parameters are fed into the system. The pulley with tension spring 208 to create frictional rotation. The pulley with 2 precision pressure sensors 209 provides two-point sliding system. The unit is powered by 12-volt power supply (210; FIG. 1-109). The fabrication unit 20 (FIG. 100) includes a collection spool with steeper motor and feedback drive system (211).

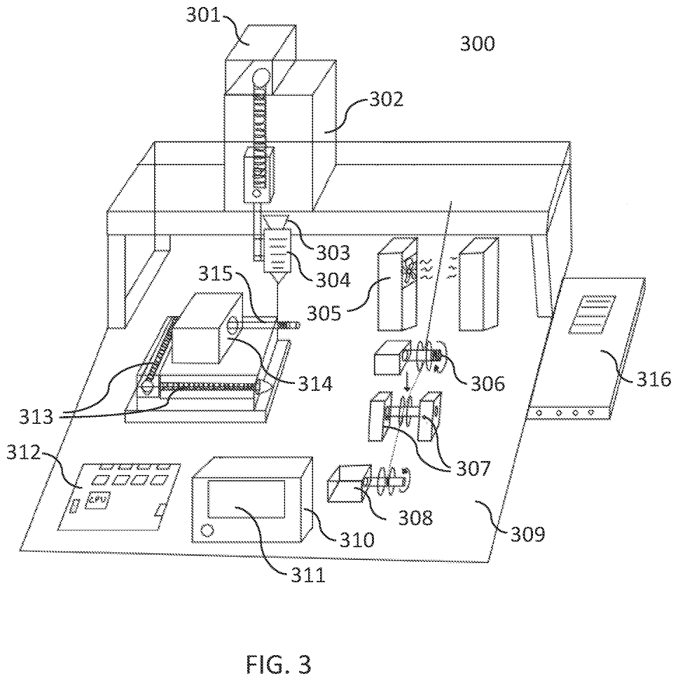

Referring to FIG. 3, a non-limiting diagram illustrates schematically the hybrid modular fabrication system 300 for making bioresorbable flow diverting device (BFDD) and continuous micrometer fiber from bioresorbable material and collected on a spool 308 as on demand. Modular unit/components dedicated for making BFDD comprise a micromotion stage 313, variable speed motor 314, and rotary arm 315. The modular unit/components for making continuous fiber comprise the cooling system 305 with integrated fan and temperature sensor, pulley with tension spring 306, sliding pulley with precision pressure sensors 307, and collection spool 308 with feedback drive system.

The power screw drive system 301 as shown in FIG. 3, provides Z-axis actuation 302 for vertical positioning. Feed material container 303 is connected with the replaceable electromelt extruder 304, which can take filament as well as powder/bead form of material to feed through the electro-melt extruder 304. The cooling system 305 has an integrated fan and temperature sensors. The replaceable electro-melt extruder 304 is connected to a feed material container 305. The pulley has a tension spring 306 to create frictional rotation. The pulley 307 with 2 precision pressor sensors provides two-point sliding system. The collection spool 308 has a steeper motor and feedback drive system. The hybrid modular fabrication system 300 is built on aluminum frame 309. The control box 310 and modular selection unit 311, and CPU 312 work as user interface where input parameters are fed into the hybrid modular fabrication system 300 and control codes are generated. The micromotion stage 313 has two degrees of freedom and it allows the stage to move in x direction and y direction. Motor with gearing system 314 provides rotational freedom. The rotary arm 315 with variable chuck can rotate either direction and can take different shape/size rotary arms using the gearing system 314. The hybrid modular fabrication system 300 is powered by 12-volt power supply 316.

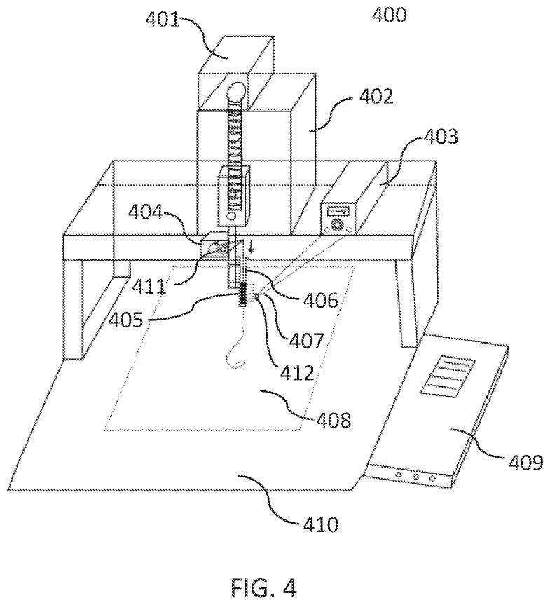

Referring to FIG. 4, a non-limiting diagram illustrates schematically the bioresorbable flow diverting device (BFDD) fabrication unit 400 mounted on an upright flatbed system. The power screw drive system 401 includes Z-axis actuation 402 for vertical positioning. Variable temperature supply source 403 provides required temperature to the heater cartridge 407 located in the aluminum feed material container 405 and push piston 406. Servo motor 404 controls the feed material rate. Replaceable flatbed 408 provides for printing upright. The flatbed 408 can be replaced with micromotion stage with rotary printing arm as shown in FIGS. 1 and 3 (106 and 313) or with the continuous microfiber making modular unit components (305, 306, 307) as shown in FIG. 3. The unit is powered by 12-volt power supply 409. The BFDD fabrication unit 400 can include a servo gear and driving system 411 to control the material deposition rate and an aluminum hot cartridge housing 412 of the fabrication system 400.

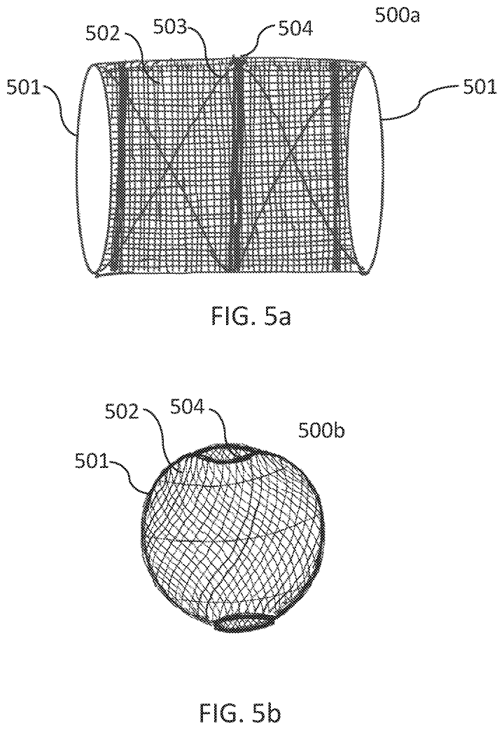

Referring to FIG. 5 a and FIG. 5b a non-limiting diagram shows schematically the elements comprising the flow diverter device (1-DD) 500 according to embodiments of the present invention with two different shapes (FIG. 5a & FIG. 5b) depending on the aneurysm types and locations. The FDD 500a comprises four elements which are diameter control ring 501, pore/scaffolding control threads 502, structural support thread/liner 503, and radiopaque coating 504. The FDD 500b comprises three elements which are diameter control ring 501, pore/scaffolding control threads 502, and radiopaque coating 504. All of these elements can be made of the same biodegradable materials (e.g., PCL) or different types of biodegradable materials or composite/blended biodegradable materials. The diameter control ring 501 determines the size of the FDD 500 depending of the parent artery or blood vessel diameter. The pore/scaffolding control threads 502 determine the pore shape, sizes, architecture and overall porosity of the FDD 500. The pore control threads 502 are very thin and can be controlled within a wide range from 30 micrometer to 100 micrometers. The structural support threads/liner 503 provides mechanical strength and flexibility to take the load/pressure exerted by the blood vessel. It also holds together the pore/scaffolding control threads 502. The mechanical strength of the FDD 500 can be increased by increasing the number of the structural support thread/liner 503. The size of the structural support thread/liner 503 can also be controlled within a wide range between 100 micrometers to 400 micrometers or as needed. Radiopaque coating (e.g., Barium sulfate, BaSO₄) provides visibility of the FDD 500 during the deployment and/or post treatment management through angiography/x-ray images. The FDD 500 can also be made without diameter control ring 501 and structural support thread 502, however, in this case, thicker microfiber (at least 50 micrometer diameters) are used.

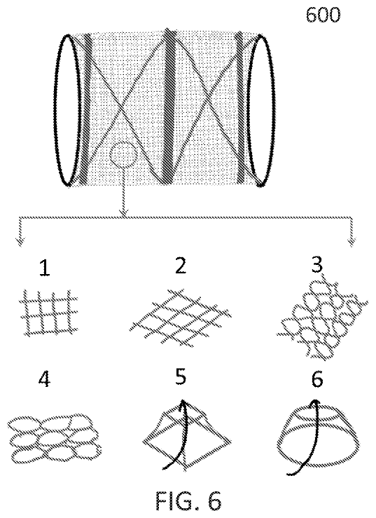

Referring to FIG. 6, a non-limiting diagram shows the non-braided surface structure of the FDD 600 provided by the present invention. In constrast to the prior art, the current or available FDDs are based on braided microwires, while the FDD 600 of the present invention is non-braided. The PCL FDD 600 of the present invention is a biodegradable material, on the other hand current FDDs use materials that are not biodegradable and are based on metallic wires. The FDD 600 of the present invention can be fabricated in at least the four pore shapes and designs (i.e., 1, 2, 3, 4) shown and are not limited to diamond shapes like in the currently available braided FDDs. Further, the cross-sections of the pore at the inlet and outlet (i.e, 5, 6) can be engineered in at least the two shapes shown, while current braided FDDs are limited to no-variation at the inlet and outlet diamond shaped pore. In addition, current braided FDDs use platinum wire for the radiopaquacity, while FDD 600 of the present invention is coated with BaSO₄ or Bismuth Nitriate for radiopaquacity.

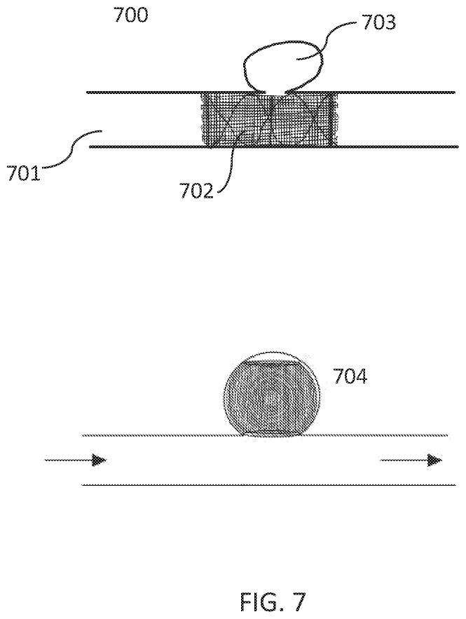

Referring to FIG. 7, a non-limiting diagram shows an embodiment of the FDD 702 the present invention placed in a parent artery 701 at the level of the aneurysm neck to disrupt the intra-aneurysmal 703. Flow diversion is accomplished when the device is placed in the parent blood artery 701 to divert blood flow away from the aneurysm itself, instead of placing a device inside the aneurysm sac, such as with current coiling techniques. Globe shaped FDD 704 is shown as deployed in the aneurysm that alters the blood flow inside the aneurysm to initiate curing without disruption of the blood flow in the parent artery. Globe shaped FDD 704 can work more efficiently in aneurysm that forms on a complex blood vessel junction.

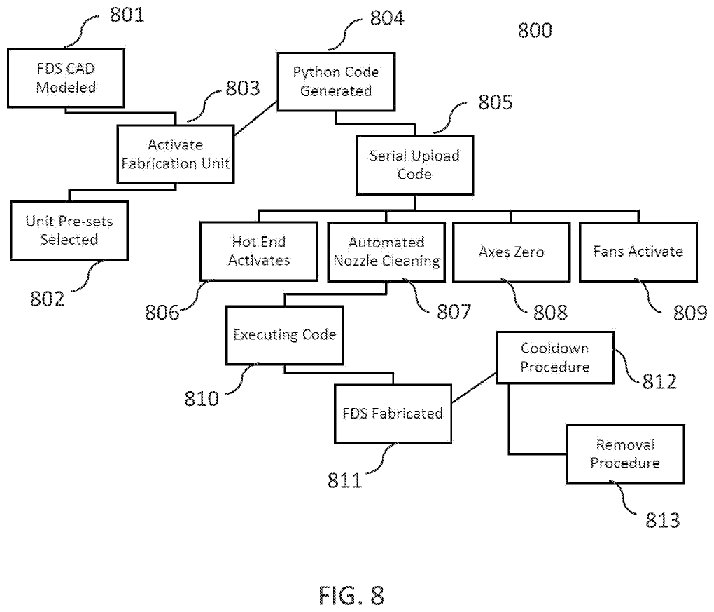

Referring to FIG. 8, a non-limiting diagram shows a process for fabricating the FDD (FIG. 6-600 and FIG. 7-700) using the fabrication unit (FIG. 1-100, FIG. 2-200) of the present invention. The process 800 of the present invention can begin with either from a CAD model 801 of BFDD in case of surface engineered BFDD (FDS CAD modeled) or based on a desired porosities and nominal diameter 802 of the BFDD input into a controller (Unit Pre-sets selected) that determines the rotational speed of the rotary arm, length of the BFDD, translational speed, number of passes of translational motion of the arm and strut thickness needed to activate 803 the system. Based on these inputs the fabrication system generates control codes 804 (e.g., python G-codes), uploads the code 805 and prepares the fabrication system for fabricating BFDD. In the preparation stage, the system sets the desired temperature in the extruder nozzle 806, cleans the nozzle by flashing 807, calibrates the position of the printing arm and checks all sensors 808, and runs the cooling fan 809. After checking and verifying all the preconditions for fabrication, the system starts 810 making BFDD on the rotary arm and stops when it finishes 811. A cool down procedure is initiated 812. Now the BFDD is ready to be removed 813 from the printing arm. In the case of fabricating continuous smooth straight microfiber, controller (FIG. 3. 310) takes the desired fiber diameter and determines the pulling tension force and rotational speed of the rotary arm (FIG. 3, 315). Then the fabrication unit 300 is activated, control codes (e.g., python G-codes) are generated 804 and uploaded 805 for rotational speed, tension force and spool winding rate. Before execution, the fabrication system checks and verifies extruder nozzle temperature 806, flashing 807 and cooling fan 809, and then starts 809 fabricating smooth, straight and continuous microfiber and collecting it on a spool 9 FIG. 3, 308). Samples of the microfiber produced by the present invention exhibited an Ra below 0.12 micrometers (microns) and have been measured with an Ra at 0.1186 and at 0.0752 micrometers.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

In the Claims,

1. An endovascular device for the treatment of aneurysm, comprising a biodegradable flow diverting device (BFDD) that regulates blood flow into an aneurysmal sac, acts as a scaffold for endothelization at the neck of an aneurysm and, degrades after successful dissolution of aneurysm and remodeling of blood vessel.

2. The endovascular device of claim 1, wherein said BFDD is non-braided and comprises a plurality of pore shapes, sizes, architectures particularly at the inlet and outlet of the pores.

3. The endovascular device of claim 1, wherein pore densities and porosities are optimized for the blood vessel and aneurysmal morphologies determined from patient MRI images.

4. The endovascular device of claim 1, wherein the BFDD is shaped on a rotary arm with programmable variable speed and diameter in conjunction with a micromotion stage.

5. A fabrication unit for making a bioresorbable flow diverter device (FDD), the fabrication unit comprising:
a power screw drive system including a Z-axis actuator that provides for vertical positioning;
a rotary arm configurable for a variable chuck that can rotate either direction and accept different shape/size rotary arms;
a micromotion stage that has two degrees of freedom and allows the stage to move in x direction and y direction,
a replaceable electro-melt extruder including a connected feed material container;
wherein said rotary arm is used to control quality of surface characteristics of said bioresorbable FDD, and
wherein said electro-melt extruder can accept filament as well as powder/bead form of material to feed through the electro-melt extruder.

6. The fabrication unit of claim 5, further comprising a control box and a CPU that work as user interface where input parameters are fed into said fabrication unit.

7. The fabrication unit of claim 5, further comprising pulley with tension spring to create frictional rotation, the pulley configured with 2 precision pressure sensors providing a two-point sliding system.

8. The fabrication unit of claim 5, further comprising a collection spool with steeper motor and feedback drive system.

9. The fabrication unit of claim 8, further comprising modular components dedicated for making BFDD comprising a micromotion stage, a variable speed motor, and a rotary arm.

10. The fabrication unit of claim 9, wherein the modular components include an integrated fan and temperature sensor, a pulley with tension spring, a sliding pulley with precision pressure sensors, and a collection spool with feedback drive system.

11. A method of fabricating a bioresorbable flow diverter device (FDD), the fabrication method comprising:
creating one of a CAD model of BFDD for surface engineered BFDD or defining parameters for porosities and nominal diameter of the BFDD;
inputting one of said CAD Model or said parameters into a controller to set the rotational speed of the rotary arm, length of the BFDD, translational speed, number of passes of translational motion of the arm and strut thickness;
generating control codes and setting the desired extruder nozzle temperature;
calibrating the position of the printing arm, and running checks of all sensors and cooling fan;
verifying all preconditions for fabrication, and initiating BFDD fabrication on a rotary arm.

12. The method of claim 11, wherein said controller sets fabrication for the desired fiber diameter to produce continuous, substantially smooth and straight microfiber, and determines the pulling tension force and rotational speed of the rotary arm required to produce said microfiber.

13. The method of claim 12, wherein the fabrication unit is activated, control codes are generated for rotational speed, tension force and spool winding rate.

14. The method of claim 13, wherein the control codes are python G-Codes. 12.

15. The method claim 14, wherein the fabrication unit can be set to continuously produce microfiber exhibiting an average surface roughness (Ra) less than 0.15 micrometer.

16. The method claim 14, wherein the fiber produced by the fabrication unit exhibits an average surface roughness (Ra) less than 0.08 micrometer.
